(19) United States
(12) Patent Application Publication
(10) Pub. No.: US 2026/0295143 A1
(43) Pub. Date: Oct. 1, 2026

(54) Coiled Catheter and Methods of Use

(71) Applicant: Bard Peripheral Vascular, Inc., Franklin Lakes, NJ (US)

(72) Inventors: Melanie Hadlock, Goodyear, AZ (US); Mary Arwen Broughton, Cary, NC (US); Mircea Despa, Cary, NC (US); Akiva Kirschner, Chicago, IL (US)

(21) Appl. No.: 19/478,662

(22) PCT Filed: Apr. 26, 2024
(86) PCT No.: PCT/US2024/026479

§ 371 Date: Oct. 24, 2025

Related U.S. Application Data
(60) Provisional application No. 63/462,045, filed on Apr. 26, 2023.

(51) Int. Cl. A61M 1/00; A61M 39/00; A61M 39/24

(52) U.S. Cl. CPC A61M 1/87; A61M 39/24; A61M 2039/0009; A61M 2202/0014; A61M 2202/04; A61M 2205/0266; A61M 2210/101; A61M 2210/1017

(57) ABSTRACT

An apparatus for managing pleural or peritoneal fluid of a patient, the apparatus includes a valve and an elongated tubular portion extending from the valve with a lumen therein extending from a proximal end of the tubular portion to a distal end of the tubular portion. The elongated tubular portion further comprises an internal portion, the internal portion configured to be inserted into the pleural or peritoneal space of the patient for removing fluid from a pleural or peritoneal space. The elongated tubular portion further comprises an external portion that is closer to the proximal end than the internal portion, the external portion configured to remain external to the patient for draining the fluid via the internal portion, wherein the external portion has a shape memory feature with a non-linear natural state.

### CROSS REFERENCE TO RELATED APPLICATIONS

The present application claims the benefit of priority to U.S. Provisional Application No. 63/462,045 entitled “ALL IN ONE DRESSING, CATHETER, AND ALL IN ONE DRESSING CATHETER SYSTEM,” filed on Apr. 26, 2023, the entirety of which is hereby incorporated by reference for all purposes.

### TECHNICAL FIELD

This disclosure is related to a tunneled indwelling catheter and methods of manufacture and use.

### BACKGROUND

Typically, the dressing procedure of a tunneled indwelling catheter involves placement of several separate components (foam pad, gauze, self-adhesive dressing) and some users and caregivers find it difficult to apply the dressing procedure component without help from another person. Further, use and maintenance of a tunneled indwelling catheter and related drainage system requires a series of clean and/or aseptic steps for pre-drainage, bottle/bag drainage, and post-drainage procedures. These steps are in place to promote care and cleanliness of the catheter. The steps required for pre-drainage and post-drainage, especially dressing and/or securing of the exit point of an indwelling catheter can be difficult to manage with only two hands.

### SUMMARY

The following presents a simplified summary of one or more aspects of the technology disclosed herein in order to provide a basic understanding of such aspects. This summary is not an extensive overview of all contemplated aspects and is intended to neither identify key or critical elements of all aspects nor delineate the scope of any or all aspects. Its sole purpose is to present some concepts of one or more aspects in a simplified form as a prelude to the more detailed description that is presented later.

Disclosed herein is an apparatus for managing pleural or peritoneal fluid of a patient, the apparatus comprising a valve and an elongated tubular portion extending from the valve with a lumen therein extending from a proximal end of the tubular portion to a distal end of the tubular portion. The elongated tubular portion further comprises an internal portion, the internal portion configured to be inserted into the pleural or peritoneal space of the patient for removing fluid from a pleural or peritoneal space. The elongated tubular portion further comprises an external portion that is closer to the proximal end than the internal portion, the external portion configured to remain external to the patient for draining the fluid via the internal portion, wherein the external portion has a shape memory feature with a non-linear natural state.

In another aspect disclosed herein, a method of making an apparatus for managing plural or peritoneal fluid of a patient is disclosed. The method comprises forming an elongated tubular portion with a first portion configured to remain external to the patient for draining the fluid of an internal portion of the elongated tubular portion, wherein the external portion is formed to have a natural shape that is non-linear.

To the accomplishment of the foregoing and related ends, the one or more aspects of the disclosure comprise the features hereinafter fully described and particularly pointed out in the claims. The following description and the annexed drawings set forth in detail include certain illustrative features of the one or more aspects. These features are indicative, however, of but a few of the various ways in which the principles of various aspects can be employed, and this description is intended to include all such aspects and their equivalents.

### BRIEF DESCRIPTION OF THE DRAWINGS

Features characteristic of aspects of the technology described herein are set forth as follows, in the appended claims, and in the drawings. In the descriptions that follow, like parts are marked throughout the specification and drawings with the same numerals, respectively. The drawing figures are not necessarily drawn to scale and certain figures can be shown in exaggerated or generalized form in the interest of clarity and conciseness. The disclosure itself, however, as well as a preferred mode of use, further objects and advances thereof, will be best understood by reference to the following detailed description of illustrative aspects when read in conjunction with the accompanying drawings.

FIG. 1 shows one example of drainage device according to aspects of the disclosure installed in a patient.

FIG. 2 shows an example of a drainage device according to aspects of the disclosure.

FIG. 3 is a schematic view of an example of a drainage device according to aspects of the disclosure.

### DETAILED DESCRIPTION

The detailed description set forth below in connection with the appended drawings is intended as a description of various configurations and is not intended to represent the only configurations in which the concepts described herein can be practiced. The detailed description includes specific details for the purpose of providing a thorough understanding of various concepts. However, it will be apparent to those skilled in the art that these concepts can be practiced without these specific details. In some instances, well known structures and components are shown in block diagram form in order to avoid obscuring such concepts.

### I. Terminology

Reference throughout this specification to one aspect, an aspect, one example or an example means that a particular feature, structure or characteristic described in connection with the embodiment or example may be a feature included in at least example of the present disclosure. Thus, appearances of the phrases in one aspect, in an aspect, one example or an example in various places throughout this specification are not necessarily all referring to the same example. Furthermore, the particular features, structures or characteristics may be combined in any suitable combinations and/or subcombinations in one or more embodiments or examples.

Throughout the disclosure, the terms substantially or approximately may be used as a modifier for a geometric relationship between elements or for the shape of an element or component. While the terms substantially or approximately are not limited to a specific variation and may cover any variation that is understood by one of ordinary skill in the art to be an acceptable level of variation, some examples are provided as follows. In one example, the term substantially or approximately may include a variation of less than 10% of the dimension of the object or component. In another example, the term substantially or approximately may include a variation of less than 5% of the object or component. If the term substantially or approximately is used to define the angular relationship of one element to another element, one non-limiting example of the term substantially or approximately may include a variation of 5 degrees or less. These examples are not intended to be limiting and may be increased or decreased based on the understanding of acceptable limits to one of skill in the relevant art.

For purposes of the disclosure, directional terms are expressed generally with relation to a standard frame of reference when the aspects or articles described herein are in an in-use orientation. In some examples, the directional terms are expressed generally with relation to a left-hand coordinate system.

Terms such as a, an, and the, are not intended to refer to only a singular entity, but also include the general class of which a specific example may be used for illustration. The terms a, an, and the, may be used interchangeably with the term at least one. The phrases at least one of and comprises at least one of followed by a list refers to any one of the items in the list and any combination of two or more items in the list. All numerical ranges are inclusive of their endpoints and non-integer values between the endpoints unless otherwise stated.

The terms first, second, third, and fourth, among other numeric values, may be used in this disclosure. It will be understood that, unless otherwise noted, those terms are used in their relative sense only. In particular, certain components may be present in interchangeable and/or identical multiples (e.g., pairs). For these components, the designation of first, second, third, and/or fourth may be applied to the components merely as a matter of convenience in the description.

The terms disposed on, disposed along, disposed with, or disposed toward and variations thereof may define one element can be integral with another element, or that one element can be a separate structure bonded to or placed with or placed near another element.

The term at, for example when referring to something being located at a specific location, is intended to include any one or more of: proximate, on, near, adjacent to or within the specific location. As used herein, the terms proximal is defined with respect to an object, element, or user. For example, the term proximal may refer to the part or portion closer to the user.

### II. Overview

This overview, and the detailed description that follows, has been presented for purposes of illustration and description. It is not intended to be exhaustive nor to limit the disclosure to the forms described. Numerous modifications are possible considering the teachings herein, including any combination of the different examples described herein. Some of those modifications have been discussed and others will be understood by those skilled in the art. The various aspects were chosen and described to best illustrate the principles of the present disclosure and various aspects as are suited to the particular use contemplated. The scope of the present disclosure is, of course, not limited to the examples or aspects set forth herein but can be employed in any number of applications and equivalent devices by those of ordinary skill in the art. Rather, it is hereby intended the scope be defined by the claims appended hereto.

Typically, after a tunneled indwelling catheter is installed, the catheter is covered with a dressing, and the dressing must be regularly replaced for sanitary reasons. Dressing a tunneled indwelling catheter involves placement of several separate components (foam pad, gauze, self-adhesive dressing) and some users and caregivers find it difficult to apply the dressing procedure component on their own. For example, to dress a catheter, a foam dressing may be placed around the catheter exit from the body, the catheter may be coiled into place, and a piece of gauze may be placed over the catheter, and then a sheet of self-adhesive dressing (e.g., 3M™ Tegaderm™) may be applied over the foam, catheter, and gauze. Many users find it difficult to accomplish this workflow without the assistance of a second person.

Use of and maintenance of an indwelling catheter and related drainage system also requires series of clean and/or aseptic steps for pre-drainage, drainage, and post-drainage procedures. These steps are in place to promote care and cleanliness of the catheter. The steps required for pre-drainage and post-drainage, especially dressing and/or securing of the exit point of an indwelling catheter can be difficult to manage with only two hands while maintaining cleanliness and/or aseptic protocol. The difficultly in managing a dressing procedure may increase the chance of user error, contamination, and/or may reduce a patient's compliance for regular drainage via the catheter and/or replacement of the catheter and/or dressing. Additionally, it may be especially difficult for a patient to dress their catheter on their own if the catheter is in a hard-to-reach location such as on the side of the chest over the ribcage and/or if the patient experiences mobility challenges.

A catheter or other apparatus for managing fluid is disclosed herein. The disclosed catheter may include features that may significantly improve ease and efficiency of applying, removing, or otherwise exchanging a catheter dressing, and may especially benefit a patient and/or technician applying the dressing or catheter management system independently, i.e., without the help of others. Further, the catheter described herein may improve the ease of covering up or otherwise protecting the portion of the catheter that extends from a patient's body, which may improve comfort, convenience and/or quality of life for a patient that has an indwelling catheter.

Further, the catheter or other apparatus for managing fluid disclosed herein includes features that may improve ease of handling the catheter during and/or after drainage, for example by providing a predictable location of the valve or port on the catheter without the need for additional fasteners and/or other features that may cause discomfort or may need to be regularly removed and/or replaced.

The aspects described herein provide a safety, comfort, usability, and/or efficiency advantage for catheter patients. In one example, the aspects described herein are usable with pleural catheter systems. In another example, the aspects described herein are usable with peritoneal catheter systems. It is noted that aspects described herein are applicable to all types of catheter systems, ports, and/or intravenous therapy systems as well.

In one example, a catheter may have an internal portion configured to be placed inside a patient to an area that requires drainage and that may require frequent drainage. The catheter further includes an outer portion that is external to the patient and that may be handled during before, during, and after a drainage procedure and/or during a dressing procedure of the catheter. In the examples described below, the external portion may include a shape memory section or pigtail that is configured to naturally spring-back to a desired state, such as a coiled or helical shape. Thus, the external portion of the catheter remains manageable and contained when no external forces are applied to the external portion of the catheter. Further, if a catheter needs to be extended or manipulated, the catheter can easily be extended or manipulated, which may improve convenience and patient comfort during a drainage procedure. Further, the aspects described herein may decrease the risk of the internal portion of the catheter being dislodged or otherwise moved unintentionally.

Additional details and examples and advantages are described in the non-limiting examples below.

### III. Detailed Examples

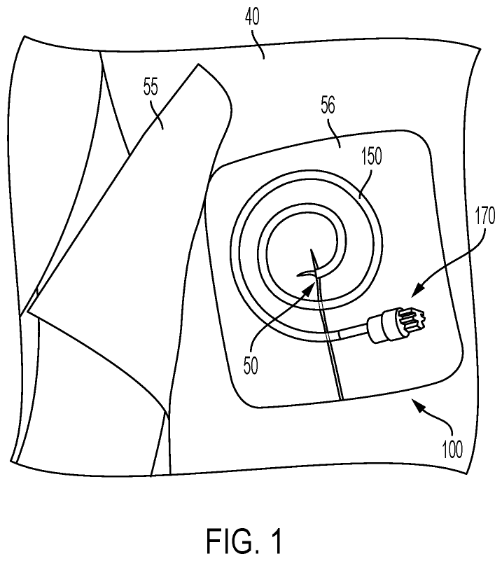

FIG. 1 shows an example of a catheter or apparatus for managing pleural fluid of a patient. As shown in FIG. 1, an indwelling catheter 100 (it is noted that throughout the disclosure the terms indwelling catheter, catheter and/or apparatus for managing fluid may be used interchangeably) of a patient includes an internal portion that is generally passed through an incision 50 in patient's skin 40 and an external portion 150 that extends from the incision. The catheter may include a valve 170 at a proximal end thereof that is configured to be capped-off when not in use and includes a valve that seals off the inner passage of the indwelling catheter 100 when not in use, but opens a fluid path when a drainage device (e.g., a drainage catheter with a dilator) is installed to the valve 170. The exit site of the indwelling catheter 100 may be dressed via an adhesive sheet or other dressing 56 with the catheter passed through an opening in the dressing 56. Further, when not in use, the exposed portion of the indwelling catheter 150 and the dressing 56 may be covered by a protective sheet 55 or other dressing that isolates the external portion 150 of the indwelling catheter 100 and the incision 50 from an external environment. Typically to dress or otherwise cover the external portion of an indwelling catheter, a user would have to coil or otherwise bundle the length of catheter that protrudes from the patient and hold the catheter while attempting to cover the catheter with the protective sheet 55. As noted above, this dressing procedure is cumbersome and results in several disadvantages that include but are not limited to the disadvantages listed above.

Aspects of the disclosure relate to an indwelling catheter that includes an external portion that has a shape memory feature or otherwise is configured to rest in a shape that is easy to deal with for a patient. In one example, the external portion of a catheter may be configured to rest in a non-linear shape (i.e., a shape other than a straight or relatively straight line). In the example shown in FIG. 1, the external portion 150 is configured to rest in a naturally coiled shape as shown in FIG. 1. For example, a length or portion of the external portion 150 may be configured to rest naturally in a spiral shape or a joined sequence of concentric circles or rings. In other words, when not subject to any external forces, the length or portion of the external portion 150 of the catheter may be spiraled as shown in FIG. 1. Further, if an external force (e.g., a straightening or pulling force) is applied to the coiled length of portion of the external portion 150 of the catheter and then released, the external portion 150 may return to a naturally coiled state. Thus, the external portion of the catheter according to an aspect of the disclosure remains compact and manageable and contained when no external forces are applied to the external portion of the catheter, which allows for the external portion 150 of the indwelling catheter 100 to remain compact and low-profile against a patient's body. Further, since the external portion 150 of the indwelling catheter 100 returns to a compact state once an external force is removed, dressing of the catheter (e.g., placement of the protective sheet 55 and/or replacement of the dressing 56 is more manageable and less prone to errors or accidents). Further, if the catheter 100 needs to be extended or manipulated, the external portion 150 can be easily straightened, extended, or otherwise manipulated, which may improve convenience and patient comfort before, during, and after a drainage procedure and/or a dressing procedure.

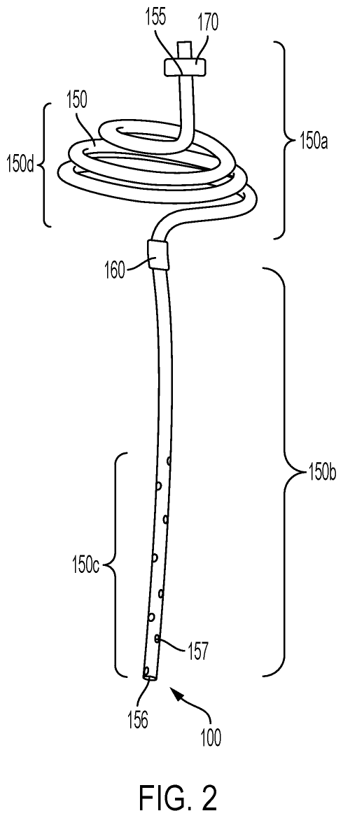

FIG. 2 shows an example non-installed view of an indwelling catheter 100 according to aspects of the disclosure. The indwelling catheter 100 may share features with or may be analogous with the indwelling catheter 100 in FIG. 1. The indwelling catheter 100 may include an external portion 150 that may generally be configured to be outside of a patient's body (e.g., as shown in FIG. 1) when installed. The indwelling catheter 100 may further include an inner portion 150b, which may be installed into a patient by a medical practitioner. The inner portion 150b typically remains inside a patient with the external portion 150 remaining external to the patient. In one example, a fenestration region 150c may include a series or plurality of fenestrations 157 that provide fluid communication with the lumen inside the catheter. Further, Further, the catheter 100 may have an opening at a distal end 156 that provides fluid communication with a valve 170 connected at a proximal end 155 of the catheter. The valve 170 at a proximal end thereof may be configured with a removable cap or other interface to cap-off a fluid communication passage of the valve 170 when not in use.

The valve 170 may further include one or more valves that seals off the inner passage of the indwelling catheter 100 when not in use, but opens a fluid path when a drainage device (e.g., a drainage catheter with a dilator) is installed to the valve 170. It is noted that while throughout the disclosure drainage is discussed, the catheter may be used for infusion or other treatments where fluid or other materials or therapeutic agents are provided from the valve 170 to the opening at the distal end 156 and/or plurality of fenestrations 157.

The indwelling catheter 100 may further include cuff 160, which may be configured to be tunneled under skin and is configured for tissue ingrowth to prevent dislodgement or to otherwise ensure the inner portion 150b remains in place and is not easily pulled out. As noted above, the external portion 150 may include a shape memory or non-linear portion 150d (the terms shape memory or non-linear may be used interchangeably throughout the disclosure). The non-linear portion 150d may be configured to rest in a non-linear shape. One example of a non-linear shape may include a shape other than straight or that is not straight when compared to the inner portion 150b. In the example shown in FIGS. 1 and 2, the external portion 150 is configured to rest in a naturally coiled shape as shown in FIGS. 1 and 2. In one example, the shape of the non-linear portion 150d may resemble a pigtail, for instance. For example, a length or portion of the external portion 150 may be configured to rest naturally in a spiral shape or a joined sequence of concentric circles or rings and when not subject to any external forces, the length of the non-linear portion 150d of the catheter 100 may be spiraled as shown in FIGS. 1 and 2. Further, if an external force (e.g., a straightening or pulling force) is applied to the coiled length of portion of the non-linear portion 150d of the catheter 100 and then released, the external portion 150 may return to a naturally coiled state.

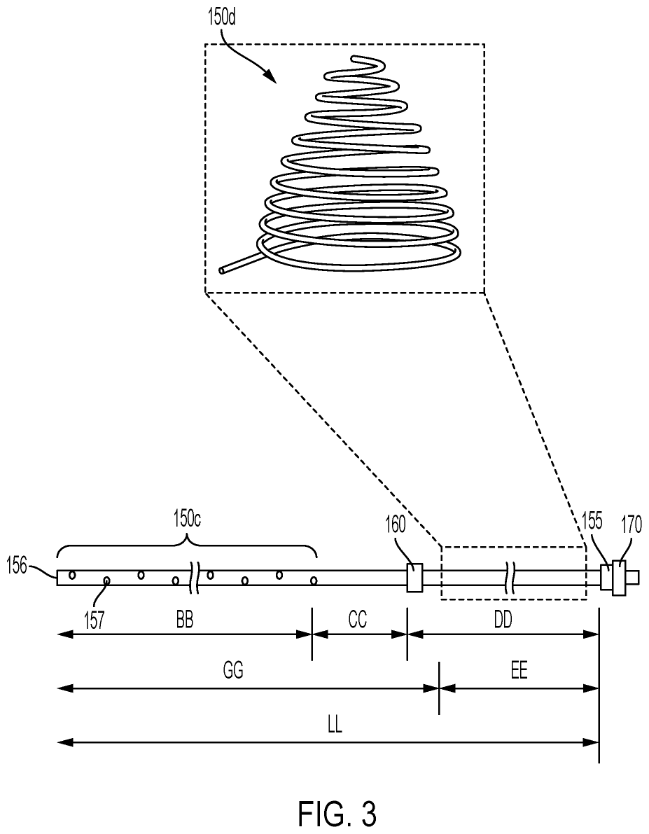

FIG. 3 shows a diagram view of a catheter according to aspects of the disclosure. The catheter of FIG. 3 may share features with or may be analogous with the catheter 100 in FIGS. 1 and 2.

As shown in FIG. 3, a catheter according to aspects of the disclosure may have an outstretched length LL (e.g., with the non-linear portion 150d stretched into a linear or straight configuration) of 55 centimeters (cm) to 150 cm, in another example, the length FF may be from 60 cm to 100 cm. In yet another example, the length FF may be approximately 66 cm.

As shown in FIG. 3, a distance from the cuff 160 to the proximal most portion of the catheter tubing 155 at the valve 170 may have an outstretched length DD (e.g., with the non-linear portion 150d stretched into a linear or straight configuration) of 10 cm to 45 cm, in another example, the length DD may be from 20 cm to 38 cm. In yet another example, the length DD may be approximately 28 cm.

The internal portion of the catheter according to aspects of the disclosure may have a length GG of 20 cm to 70 cm, in another example, the length GG may be from 35 cm to 55 cm. In yet another example, the length GG may be approximately 40 cm.

The fenestration portion 150c of the catheter according to aspects of the disclosure may have a length BB of 5 cm to 50 cm, in another example, the length BB may be from 15 cm to 40 cm. In yet another example, the length BB may be approximately 25.5 cm.

A length of the external portion and/or non-linear portion 150d may have a length EE (e.g., with the non-linear portion 150d stretched into a linear or straight configuration) of 12 cm to 40 cm, in another example, the length EE may be from 15 cm to 30 cm. In yet another example, the length EE may be approximately 26 cm.

The catheters described herein may be made of any know material for example, any one or combination of a latex, silicone, vinyl or polyvinyl chloride (PVC), Thermoplastic Polyurethane (TPU) and/or Polytetrafluoroethylene (PTFE, Teflon) to name a few examples. In some examples, either the linear portion 150b and/or the non-linear portion 150a (or both) may have visualization features to provide for x-ray or CT scan visualization. In some examples, a barium stripe may extend along the length of either the non-linear portion 150a, the linear portion 150b or both to help with x-ray or CT scan visualization. In some examples, the non-linear portion 150d may for example be formed or extruded in a tensioned or pre-formed shape so that the non-linear portion 150d naturally returns to the non-linear or coiled shapes described herein. In another example, at least the non-linear portion 150d may include or have embedded therein a shape memory material, such as an alloy, thermoplastic, and/or composite. In one example, the non-linear or coiled shape section external portion 150 may have embedded therein or included therein one or more nitinol wire and/or a heat set coil within a flexible extrusion. The non-linear portion 150d may be formed or otherwise configured so that uncoiling or overcoming the natural tendency of the non-linear portion 150d to retain its shape may require enough force so that the non-linear portion 150d remains coiled when installed in a patient, but so that overcoming the natural tendency of the non-linear portion 150d to retain its shape (e.g., to straighten the non-linear portion 150d or otherwise) would not require such a large force that a patient may be injured and/or the cuff 160 could potentially be pulled out of the incision through which the catheter passes. In some example, the durometer of the material can be optimized to provide the aforementioned desired qualities and/or to provide an optimal balance between flexibility and rigidity. In one non-limiting example, the linear portion 150b and/or the non-linear portion 150a may have a durometer between 55-95 Shore A.

In some examples, the use of or installation of the catheter 100 may be described in instructions provided with the catheter and any additional components included therewith (e.g., a catheter, dressing, and/or cleaning/sterilization supplies). The instructions may be provided as a pamphlet or as written instructions on the packaging of the indwelling catheter 100 or kit including the indwelling catheter 100. The aforementioned instructions may also be provided as digital content (provided by accessing information on a network, such as a webpage on the Internet). In one example, the indwelling catheter, packaging of the catheter and/or any components provided therewith may include an address and/or a quick response “QR” code for accessing instructions and/or digital content related to methods of use thereof. In addition, the instructions may also be provided by salesperson via marketing materials to name another non-limiting example.

The drainage system described above, for example the catheter 100 may come pre-packaged in a sealed pouch, optionally with any additional components or supplies that are helpful during a drainage procedure. In one aspect, the catheter 100 may be packaged in a sealed pouch and sterilized or high-level disinfected, thus ensuring the contents remain sterilized or high-level disinfected until a user opens the pouch.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein but is to be accorded the full scope consistent with the language claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described herein that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

1. An apparatus for managing pleural or peritoneal fluid of a patient, the apparatus comprising:
a valve;
an elongated tubular portion extending from the valve with a lumen therein extending from a proximal end of the tubular portion to a distal end of the tubular portion, wherein the elongated tubular portion further comprises:
an internal portion, the internal portion configured to be inserted into the pleural or peritoneal space of the patient for removing fluid from a pleural or peritoneal space; and
an external portion that is closer to the proximal end than the internal portion, the external portion configured to remain external to the patient for draining the fluid via the internal portion, wherein the external portion has a shape memory feature with a non-linear natural state.

2. The apparatus of claim 1, wherein the non-linear natural state is a coiled or helical state.

3. The apparatus of claim 1, wherein the internal portion is configured in a linear natural state.

4. The apparatus of claim 1, wherein the internal portion has a series of fenestrations that provide fluid communication with the lumen.

5. The apparatus of claim 1, wherein the internal portion extends between 15 centimeters (cm) to 35 cm from the distal end of the elongated tubular portion.

6. The apparatus of claim 1, wherein the external portion extends from the valve at the proximal end of the elongated tubular portion and is between 20 cm and 40 cm long.

7. The apparatus of claim 1, further comprising a cuff between the internal portion and the external portion.

8. The apparatus of claim 1, wherein the elongated tubular portion is formed of a flexible material with a shape memory alloy embedded within walls of the flexible material at the external portion.

9. The apparatus of claim 8, wherein the shape memory alloy is helical or coiled in a natural state.

10. The apparatus of claim 1, wherein the external portion of the tubular portion is molded in a non-linear shape.

11. The apparatus of claim 1, wherein the valve is at proximal end of the tubular portion.

12. The apparatus of claim 1, wherein the valve is configured open when engaged with a drainage apparatus for removal of fluid from the pleural or peritoneal space via the elongated tubular portion and wherein the valve is configured to close when the drainage apparatus is disengaged from the valve.

13. The apparatus of claim 1, wherein the valve is configured to receive a drainage apparatus for removal of fluid from the pleural or peritoneal space via the elongated tubular portion.

14-15. (canceled)
